(19) United States
(12) Patent Application Publication
(10) Pub. No.: US 2013/0061857 A1
(43) Pub. Date: Mar. 14, 2013

(54) SURGICAL RETAINING CLAMP AND METHODS OF USE

(71) Applicants: David McNally, Salt Lake City, UT (US); Mark Stringham, Salt Lake City, UT (US); Ed Hammond, Salt Lake City, UT (US)

(72) Inventors: David McNally, Salt Lake City, UT (US); Mark Stringham, Salt Lake City, UT (US); Ed Hammond, Salt Lake City, UT (US)

(21) Appl. No.: 13/605,879

(22) Filed: Sep. 6, 2012

Related U.S. Application Data
(60) Provisional application No. 61/532,506, filed on Sep. 8, 2011.

(51) Int. Cl. A61B 19/10; F16L 3/12

(52) U.S. Cl. 128/852; 248/49

(57) ABSTRACT

A clamp for securing a surgical instrument or tubing to surgical drapes is provided. The clamp may be attached to surgical drapes and include a retaining member for engaging the cable of a surgical instrument or tubing. The clamp secures the surgical instrument or tubing to the surgical drapes so as to prevent the surgical instrument or tubing from falling outside the sterile field while allowing for convenient repositioning of the surgical instrument when desired. The clamp may also include an attachment member to minimize the risk of tearing of the surgical drapes when the clamp is attached thereto. Additionally, the clamp may include a gripping member that allows the user to operate the clamp while using surgical gloves.

### RELATED APPLICATIONS

The present application claims the benefit of U.S. Provisional Patent Application Ser. No. 61/532,506, filed Sep., 08, 2011, which is incorporated herein by reference in its entirety.

### THE FIELD OF THE INVENTION

The present invention relates to surgical clamps. More specifically, the present invention relates to a clamp for securing a surgical instrument to surgical drapes to ensure that the surgical instrument stays within the sterile field. The clamp may also be used to secure other structures such as tubing and electrical cords to surgical drapes during a procedure.

### BACKGROUND

Proper aseptic technique is one of the most fundamental and essential principles of infection control in the clinical and surgical setting. The word “aseptic” is defined as “without microorganisms,” and aseptic technique refers to specific practices which reduce the risk of post-surgical infections in patients by decreasing the likelihood that infectious agents will invade the body during clinical and surgical procedures. These practices also are designed to help health care providers avoid being exposed to blood, body fluids, tissue and other potentially infectious material during clinical and surgical procedures.

Creating and maintaining a sterile field is an essential component of aseptic technique. A sterile field is an area created by placing sterile surgical drapes around the patient's surgical site and on the stand that will hold sterile instruments and other items needed during surgery. Areas below the level of the draped patient are considered non-sterile. A break in the sterile field during a surgical procedure may increase the risk that the patient will develop a post-surgical infection, a leading cause of patient morbidity and mortality.

Surgical instruments are also carefully sterilized prior to use in a procedure. Once a sterile object comes in contact with a non-sterile object, surface or person, however, the object is no longer sterile. For example, during a procedure a surgical instrument may be mishandled, or placed on the surgical drapes when not being used. If the surgical instrument rolls or slides off the patient or falls below the level of the draped patient, then the surgical instrument is no longer sterile. Introduction of even one non-sterile object into the sterile field may contaminate the entire filed. Thus, if the surgical instrument is dropped it cannot be used without increasing the risk that the patient will develop an infection.

Various methods have been used to help keep the surgical instrument within the sterile field after it is placed on the surgical drapes. For example, one method involves looping the cord of a surgical instrument through one or more finger loops of a towel clip or hemostat that has been attached to the surgical drapes. Thus, if the surgical instrument starts to roll or slide off of the drapes, the portion of the cord retained in the towel clip or hemostat prevents the surgical instrument from falling out of the sterile field. One drawback of this method is that the towel clip or hemostat may cause tears in the surgical drapes, thereby breaking the sterile field. Also, repositioning the surgical instrument may be inconvenient because to do so the cord must be removed and then repositioned in the towel clip or hemostat.

Thus, there is a need for a clamp for securing a surgical instrument to surgical drapes to ensure that the surgical instrument stays within the sterile field. Such a clamp should minimize the risk of tearing of the surgical drapes while allowing convenient repositioning of the surgical instrument when desired.

### SUMMARY OF THE INVENTION

It is an object of the present invention to provide a surgical instrument retaining clamp and methods of using the same.

According to one aspect of the present invention, a clamp for securing a surgical instrument cable to surgical drapes may include one or more arms for attaching the clamp to surgical drapes, and a retaining member for engaging the cable of a surgical instrument.

According to another aspect of the present invention, the retaining member may engage the cable of a surgical instrument with sufficient force to hold the cable and prevent the clamp from sliding along the cable during normal use. The force holding the cable into engagement with the retaining member, however, should not prevent a user from intentionally sliding the clamp along the cable when repositioning of the surgical instrument relative to the clamp is desired.

According to another aspect of the present invention, a clamp for securing a surgical instrument cable to surgical drapes may include a biasing member to bias one or more arms of the clamp.

According to still another aspect of the present invention, at least one arm of a retaining clamp may include an attachment member, such as teeth, knurling, etc. The attachment member may increase the strength in which the clamp engages the surgical drapes until the clamp's removal is desired. The attachment member may also substantially prevent tearing of the surgical drapes when the clamp is attached thereto.

According to yet another aspect of the present invention, at least one arm of a clamp for securing a surgical instrument cable to surgical drapes may include a gripping member which allows the user to operate the clamp while using surgical gloves.

According to another aspect, the clamp of the present invention may be used to secure surgical tubing to the surgical drapes.

These and other aspects of the present invention are realized in a clamp for securing a surgical instrument cable or tubing to surgical drapes as shown and described in the following figures and related description.

### BRIEF DESCRIPTION OF THE DRAWINGS

Various embodiments of the present invention are shown and described in reference to the numbered drawings wherein:

FIG. 1 shows a perspective view of a clamp for securing a surgical instrument cable or tubing to surgical drapes according to principles of the present invention;

FIG. 2 shows a side view of the clamp of FIG. 1;

FIG. 3 shows a perspective view of a clamp of the present invention securely holding a surgical instrument to surgical drapes;

FIG. 4 shows side view of an alternate clamp for securing a surgical instrument cable to surgical drapes according to principles of the present invention; and

FIG. 5 a top view of the clamp of FIG. 4 engaging the cable of a surgical instrument.

It will be appreciated that the drawings are illustrative and not limiting of the scope of the invention which is defined by the appended claims. The embodiments shown accomplish various aspects and objects of the invention. It is appreciated that it is not possible to clearly show each element and aspect of the invention in a single figure, and as such, multiple figures are presented to separately illustrate the various details of the invention in greater clarity. Similarly, not every embodiment need accomplish all advantages of the present invention.

### DETAILED DESCRIPTION

The invention and accompanying drawings will now be discussed in reference to the numerals provided therein so as to enable one skilled in the art to practice the present invention. The drawings and descriptions are exemplary of various aspects of the invention and are not intended to narrow the scope of the appended claims.

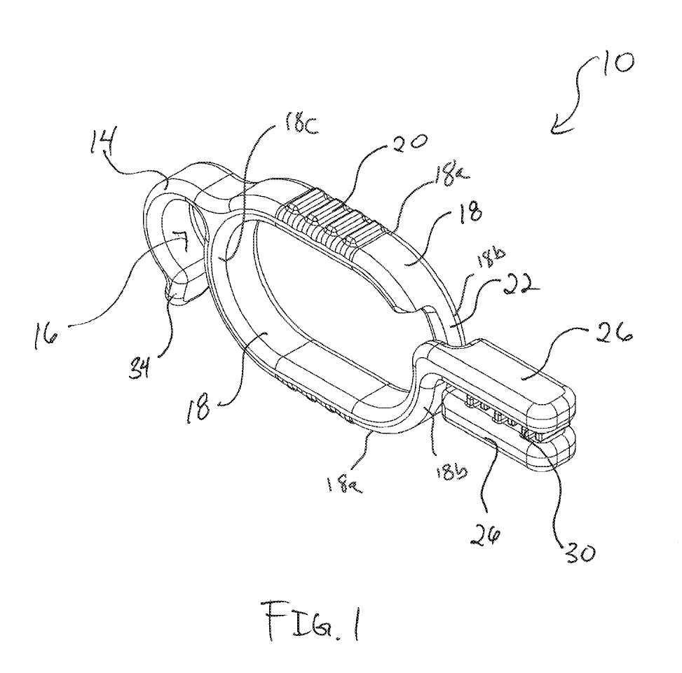

Turning now to FIG. 1, a perspective view of a clamp, generally indicated at 10, for securing a surgical instrument cable or tubing to surgical drapes is shown. The clamp 10 may include arms 18 having engagement segments 26 which extend generally parallel to each other for attaching the clamp 10 to surgical drapes. As shown in FIG. 1, the arms 18 each have a first portion 18a which is spaced apart from the other arm and a second portion 18b which overlaps the other arm prior to the engagement segments 26 so that the engagement segment of each arm is disposed opposite of the overlap from the first portion.

The arms 18 may be biased to push the first portion toward one another by application of force to the outer surface of the arms 18. Squeezing the first portion 18a of the arms 18 toward one another moves the engagement segments 26 near the ends of the arms away from each other to thereby open engagement segments 26 so that clamp 10 may be attached to the surgical drapes.

As will be appreciated, both arms 18 and the engagement segments 26 can be formed from a single piece of material. Thus, as shown in FIG. 1, the first portion of the arms 18 may be connected at an end 18c opposite the engagement segments 26 and extend, at least for a portion thereof, arcuately away and then back toward one another until they overlap and form the engagement segments 26. Being formed from one piece of material reduces production costs and reduces the number of parts which can fail.

Additionally, the engagement segments 26 may include an attachment member 30, such as teeth, knurling, etc., to enhance engagement of the clamp 10 with the surgical drapes until the clamp's removal is desired. The attachment member 30 may also substantially prevent tearing of the surgical drapes when the clamp 10 is attached thereto. The attachment member 30 may also be formed integrally with the engagement segments 26 and the remainder of the arms.

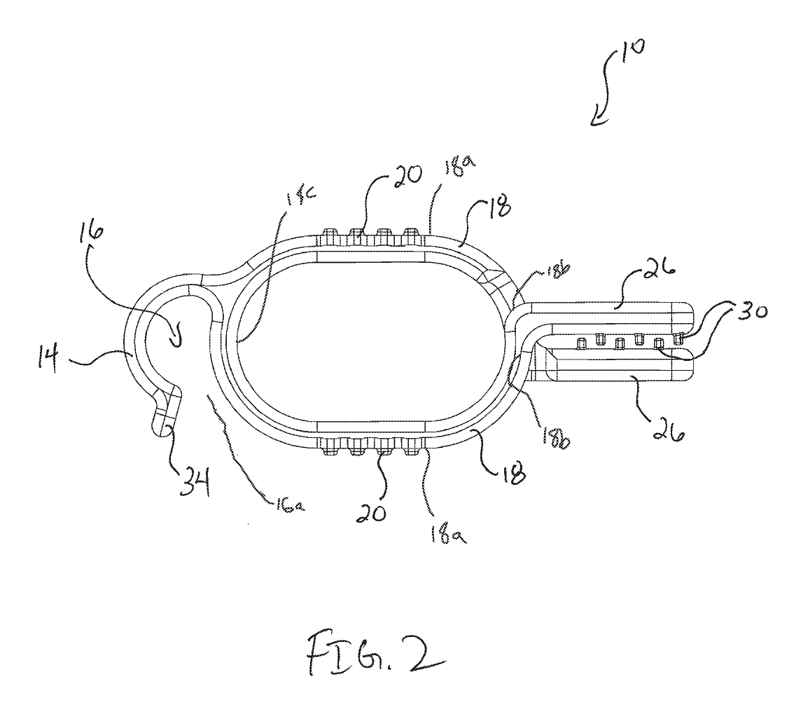

The clamp 10 may include a retaining member 14 having an opening 16 for engaging the cable of a surgical instrument or tubing when the clamp 10 is attached to the surgical drapes. The retaining member 14 may be formed integrally with the remainder of the clamp 10. The retaining member 14 may be sufficiently flexible to allow the narrow neck 16a (FIG. 2) of opening 16 to widen so that a cable of a surgical instrument or tubing may be disposed in opening 16. After the cable or tubing is disposed in opening 16 the retaining member 14 may be released so that narrow neck 16a (FIG. 2) of opening 16 returns to substantially its original position. When the retaining member 14 engages, for example, the cable of a surgical instrument, it does so with sufficient force to hold the cable in opening 16 to prevent the clamp 10 from sliding along the cable during normal use. A user of the clamp 10, however, may overcome the force when repositioning of the surgical instrument is desired and slide the clamp 10 along the cable. Additionally, the retaining member 16 may include a projection 34 to facilitate flexing of the retaining member 14 when disposing the cable or tubing in, or removing the cable or tubing from, the opening 16.

The clamp 10 may also include at least one gripping member 20, which may be disposed, for example, on the first portion 18a of the arms 18. The gripping member 20 may allow the user to operate the clamp 10 while using surgical gloves. It will be appreciated that often the surgical gloves being worn by a user of the clamp 10 will have blood or other body fluids on the outer surface of the gloves, which may make gripping the clamp 10 difficult in the absence of the gripping member 20. Thus, the gripping members 20 may facilitate removal or repositioning of the clamp 10 at any time during a surgical procedure.

The clamp 10 may be injection molded such that the arms 18 are formed as a single unit. When formed as a single unit, arms 18 may include a segment 22 that allows the arms 18 to be biased toward each other when a force is applied to the outer surface of the arms 18, and for the arm segments 26 to be biased toward each other when an external force is not applied. As will be explained in more detail below, the clamp may comprise separately formed arms 18 which are biased toward each other using a biasing member.

According to one aspect of the invention the clamp 10 may be formed using materials suitable for sterilization, such as would be achieved using an autoclave, ethylene oxide (EO) gas or gamma rays, to ensure that that the clamp 10 does not contaminate the sterile field. For example, the clamp 10 may be formed using pliable metal or plastic, such as nylon, polyethylene, polystyrene, etc.

Turning now to FIG. 2, a side view of the clamp 10 of FIG. 1 is shown. As can be more clearly seen, the arm segments 26 may include several attachment members 30 comprised of teeth or other textured surface. The attachment members 30 increase the frictional force between the surgical drapes and the arm segments 26 when the clamp 10 is attached to the surgical drapes. Thus, the attachment members 30 ensure that the clamp 10 remains securely attached to the surgical drapes during the procedure.

Additionally, the attachment members 30 may be configured to minimize the risk of tearing of the surgical drapes when the clamp 10 is attached thereto. For example, the teeth, etc., comprising the attachment members 30 in FIG. 2 may have somewhat rounded edges. Alternatively, the attachment members 30 may be comprised of knurling or other structures that also minimizes the risk of tearing of the surgical drapes.

Although the clamp 10 shown in FIG. 2 includes arms 18 both having a gripping member 20, it will be appreciated that only a single gripping member 20 may be necessary to facilitate easy use of the clamp 10 by medical personnel. In other words, a clamp 10 having a gripping member 20 on only one arm 18 may be sufficient to allow the user to easily grasp the clamp 10 while wearing surgical gloves which have blood or other body fluids on the outer surface of the gloves.

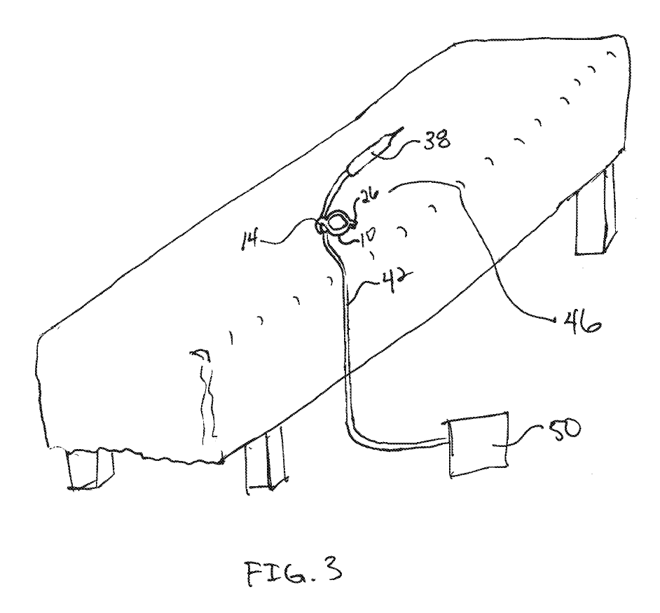

Turning to FIG. 3, there is shown a perspective view of a clamp 10 of the present invention securely holding a surgical instrument 38 to surgical drapes 46. It is becoming more common to use surgical instruments, such as surgical instrument 38, which require a power source 50 and a cable 42 linking the surgical instrument 38 to the power source 50 during a surgical procedure. By securing clamp 10 to the surgical drapes 46 and disposing cable 42 in retaining member 14, the clamp 10 may prevent the surgical instrument 38 from falling outside the sterile field when the surgical instrument 38 is not being used or it is accidentally mishandled.

Likewise, the clamp 10 may also be used to attach other tubing to the surgical drapes 46 during a procedure to securely hold the tubing within the sterile field. Use of clamp 10 to hold tubing to the surgical drapes 42 may be preferential to other methods that are currently used, such as taping tubing to the surgical drapes 46. The clamp 10 can be quickly attached to the drapes 46 and repositioned when desired. When the tubing is attached to the drapes 46 using tape, however, repositioning the tubing during a procedure may require bringing a new strip of tape into the sterile field to ensure that the tubing is securely attached to the drapes 46. This may be more time consuming than if the clamp 10 was used and increases the risk of a break in the sterile field.

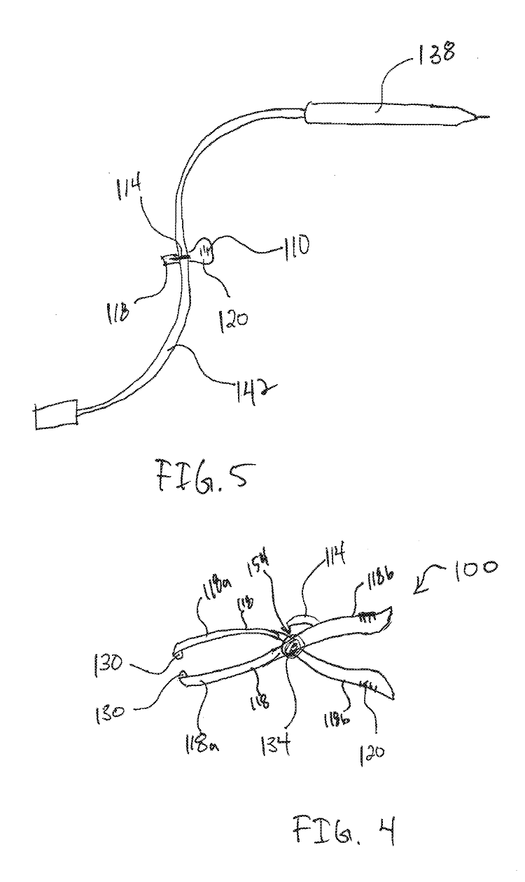

Referring now to FIG. 4, there is shown a side view of an alternate clamp, generally indicated at 100, for securing a surgical instrument cable to surgical drapes made in accordance with principles of the present invention. The clamp 100 may include separately formed arms 118 which may be operatively connected at a pivot point 154. The clamp 100 may also include a biasing member 134, such as a spring. The biasing member 134 may bias the arms 118 so that a first end of the arms 118a are substantially adjacent each other without application of an external force to the clamp. A user of the clamp 100 may apply a force a second end of the arms 118b to open the clamp 100 to move the first ends 118a away from each other temporarily for attachment to the surgical drapes.

The clamp 100 may include a retaining member 114 for engaging the cable of a surgical instrument or tubing while the clamp 100 is attached to the surgical drapes. The retaining member 114 may be sufficiently flexible to allow the cable or tubing to be disposed in engagement with the retaining member 114. For example, FIG. 5 shows the retaining member 114 engaging a cable 142 for a surgical instrument 138. The retaining member 114 may engage the cable with sufficient force to prevent the clamp 100 from sliding along the cable 142 during normal use. However, the force may be overcome by a user of the clamp 100 when repositioning of the surgical instrument is desired by sliding the clamp 100 along the cable.

Similar to the clamp 10 of FIGS. 1 and 2, the clamp 100 shown in FIG. 4 may include an attachment member 130, such as teeth, other projections, etc., which are configured to substantially prevent tearing of the surgical drapes when the clamp 100 is attached to the surgical drapes. Also, the attachment members 130 may enhance the engagement of the clamp 100 with the surgical drapes until the clamp's removal is desired.

It will be appreciated that various aspect of the invention may be used together. Thus, a clamp according to the present invention may include one or more arms for attaching the clamp to surgical drapes and a retaining member for removably engaging a structure to thereby secure the structure to the surgical drapes. It may also include a structure removably engaged by the retaining member comprising the cable of a surgical instrument; a structure removably engaged by the retaining member comprising tubing; a retaining member which prevents the clamp from sliding along the structure during normal use; a clamp which is slidable along the cable when a sufficient force is applied to the clamp; at least one attachment member, wherein the at least one attachment member enhances the attachment of the clamp to the surgical drapes; at least one gripping member; a biasing member for biasing the one or more arms into engagement with the surgical drapes when the clamp is attached to the surgical drapes; a retaining member which is flexible; a clamp being formed from materials suitable for sterilization in an autoclave, ethylene oxide (EO) gas or gamma rays, or any combination thereof.

A method of securing a cable of a surgical instrument to surgical drapes may include the steps of selecting a clamp comprising one or more arms and a retaining member for engaging the cable of the surgical instrument; biasing the one or more arms of the clamp so that the arms can be removably attached to the surgical drapes; disposing the cable of the surgical instrument in the retaining member; and attaching the clamp to the surgical drapes.

The method may also include the retaining member preventing the clamp from moving relative to the cable unless a sufficient force is applied to the clamp; the clamp being slidably engaged with the cable when a sufficient force is applied to the clamp; the clamp further comprising at least one attachment member configured to minimize the risk of tearing the surgical drapes when the clamp is attached thereto, or any combination thereof.

According to the present invention, a clamp may also include a first arm and a second arm, each arm having a first portion whereat the first arm and second arm are spaced away from each other, a second portion wherein the first arm and the second arm overlap each other, and an engagement segment disposed on the opposite side of the second portion from the first portion, the first arm and the second arm being configured to bias the engagement segment into engagement with one another; and a retaining member connected to at least one of the arms, the retaining member defining an opening and being configured for receiving and holding a tube or cable.

There is thus disclosed an improved surgical instrument retaining clamp and methods of using the same. It will be appreciated that numerous changes may be made to the present invention without departing from the scope of the claims.

What is claimed is:

1. A clamp comprising:
one or more arms for attaching the clamp to surgical drapes; and
a retaining member for removably engaging a structure to thereby secure the structure to the surgical drapes.

2. The clamp of claim 1, wherein the structure removably engaged by the retaining member comprises the cable of a surgical instrument.

3. The clamp of claim 1, wherein the structure removably engaged by the retaining member comprises tubing.

4. The clamp of claim 1, wherein the retaining member prevents the clamp from sliding along the structure during normal use.

5. The clamp of claim 4, wherein the clamp is slidable along the cable when a sufficient force is applied to the clamp.

6. The clamp of claim 1, further comprising at least one attachment member, wherein the at least one attachment member enhances the attachment of the clamp to the surgical drapes.

7. The clamp of claim 1, further comprising at least one gripping member.

8. The clamp of claim 1, further comprising a biasing member for biasing the one or more arms into engagement with the surgical drapes when the clamp is attached to the surgical drapes.

9. The clamp of claim 1, wherein the retaining member is flexible.

10. The clamp of claim 1, wherein the clamp is formed from materials suitable for sterilization, such as would be achieved in an autoclave, by ethylene oxide (EO) gas, or gamma rays.

11. A method for securing a cable of a surgical instrument to surgical drapes, the method comprising the steps of:
selecting a clamp comprising:
one or more arms; and
a retaining member for engaging the cable of the surgical instrument;
biasing the one or more arms of the clamp so that the arms can be removably attached to the surgical drapes;
disposing the cable of the surgical instrument in the retaining member;
attaching the clamp to the surgical drapes.

12. The method of claim 11, wherein the retaining member prevents the clamp from moving relative to the cable unless a sufficient force is applied to the clamp.

13. The method of claim 12, wherein the clamp is slidably engaged with the cable when a sufficient force is applied to the clamp.

14. The method of claim 11, wherein the clamp further comprises at least one attachment member, and wherein the attachment member is configured to minimize the risk of tearing the surgical drapes when the clamp is attached thereto.

15. A clamp comprising a first arm and a second arm, each arm having a first portion whereat the first arm and second arm are spaced away from each other, a second portion wherein the first arm and the second arm overlap each other, and an engagement segment disposed on the opposite side of the second portion from the first portion, the first arm and the second arm being configured to bias the engagement segment into engagement with one another; and
a retaining member connected to at least one of the arms, the retaining member defining an opening and being configured for receiving and holding a tube or cable.
